(19) United States
(12) Patent Application Publication
(10) Pub. No.: US 2026/0215844 A1
(43) Pub. Date: Jul. 30, 2026

(54) MEDICAL DEVICE HAVING EXPANSION BODY

(71) Applicant: TERUMO KABUSHIKI KAISHA, Tokyo (JP)

(72) Inventor: Yusuke TAKAHASHI, Hadano-shi (JP)

(73) Assignee: TERUMO KABUSHIKI KAISHA, Tokyo (JP)

(21) Appl. No.: 19/629,283

(22) Filed: Mar. 26, 2026

Related U.S. Application Data
(63) Continuation of application No. PCT/JP2024/032324, filed on Sep. 10, 2024.

(30) Foreign Application Priority Data
Sep. 26, 2023 (JP) 2023-163538

(51) Int. Cl. A61B 18/14; A61B 18/00

(52) U.S. Cl. CPC A61B 18/1492; A61B 2018/00178; A61B 2018/00184; A61B 2018/00267; A61B 2018/1475

(57) ABSTRACT

A medical device includes an expansion body including multiple linear bodies, a shaft portion to which a connection portion disposed on a proximal end of the expansion body is fixed, and multiple electrode assemblies each including an electrode unit and a long wiring portion including the electrode unit at the distal part thereof. The expansion body includes a recess recessed radially inward. The recess includes a bottom, a proximal-side upright portion, and a distal-side upright portion. The electrode unit is disposed along the proximal-side upright portion or the distal-side upright portion. The shaft portion includes a distal extension portion extending from the connection portion to a distal side. A distal covering portion for bundling the multiple wiring portions of the multiple electrode assemblies on the outer surface of the distal extension portion is provided between the connection portion and a distal end of the distal extension portion.

### CROSS-REFERENCES TO RELATED APPLICATIONS

This application is a continuation of International Patent Application No. PCT/JP2024/032324 filed on September 10, 2024, which claims priority to Japanese Patent Application No. 2023-163538 filed on September 26, 2023, the entire content of both of which is incorporated herein by reference.

### TECHNOLOGICAL FIELD

The present invention relates generally to a medical device that applies energy to a biological tissue.

### BACKGROUND DISCUSSION

A known device for performing an ablation treatment to cauterize biological tissue by a high-frequency current includes an expansion body configured to be expanded and contracted in a living body, and on which is disposed an electrode unit. One such treatment by ablation involves a shunt treatment on the atrial septum. Such a shunt treatment can alleviate heart failure symptoms of a patient with heart failure by forming a shunt (communication hole) serving as an escape route for an increased atrial pressure in the fossa ovalis of the atrial septum of the patient. In this shunt treatment, the atrial septum is accessed using an intravenous approaching method, and a shunt with a desired size is formed.

WO 2019-085841 discloses an expansion body that has a recess that is recessed radially inward during expansion of the expansion body to define a reception space capable of receiving the biological tissue, and can hold the biological tissue from both sides in the thickness direction. The electrode units are disposed in the recess. The electrode units constitute an electrode assembly with an elongated wiring portion for supplying power to the electrode units.

### SUMMARY

In order to dispose many electrode units on the expansion body in the circumferential direction, the expansion body has a complicated shape in which linear bodies constituting the expansion body are branched and joined along the length direction. In this case, it is difficult to dispose the wiring portions along the linear bodies. Therefore, the wiring portions are provided as separate components from the linear bodies and are disposed so as to extend from the inside of the expansion body toward the recess. When the wiring portions of the electrode assemblies are disposed as separate components from the linear bodies of the expansion body as described above, the expansion body may contract in a state where the wiring portions are caught in the gaps between the linear bodies when the expansion body expanded in the radial direction is drawn into the sheath and stored. As a result, the wiring portions may be broken due to the interference between the linear bodies and the wiring portions.

The medical device disclosed here is capable of inhibiting interference of a linear body with a wiring portion at the time of contraction in an expansion body in which the linear body and the wiring portion of an electrode assembly are provided as separate components.

A medical device disclosed here includes: an expansion body including a plurality of linear bodies and expandable and contractible in a radial direction; a shaft portion that includes a connection portion connected to a proximal part of the expansion body and that is elongated; and at least one electrode assembly including an electrode unit and a wiring portion that is elongated and that is connected to the electrode unit, wherein the expansion body includes a recess recessed radially inward when the expansion body is expanded, and is formed in such a manner that a distance between linear bodies adjacent to each other in a circumferential direction among the plurality of linear bodies constituting the expansion body changes according to expansion and contraction of the expansion body, the recess includes a bottom located on an innermost side in the radial direction, a proximal-side upright portion extending from a proximal end of the bottom toward a proximal-side top portion on a radially outer side, and a distal-side upright portion extending from a distal end of the bottom toward a distal-side top portion on a radially outer side, the electrode unit is disposed along the proximal-side upright portion or the distal-side upright portion so as to be exposed to an outside of the expansion body, the shaft portion includes a distal extension portion that is hollow and that extends from the connection portion to a distal side within the expansion body, the wiring portion of the at least one electrode assembly includes a first end connected to the electrode unit, a second end on an opposite side of the first end, and a suspended line portion disposed between the first end and the second end, the distal extension portion includes a retaining portion between the connection portion of the shaft portion and a distal end of the distal extension portion, the retaining portion retaining a section of the wiring portion proximal of the suspended line portion on the distal extension portion on an outside of an inner peripheral surface of the distal extension portion, and the suspended line portion of the wiring portion extends from the retaining portion of the distal extension portion to the proximal-side upright portion or the distal-side upright portion through an interior of the expansion body, and is aerially positioned within the expansion body in an expanded state of the expansion body.

In the medical device configured as described above, the wiring portion is retained by the retaining portion in the interior of the expansion body on the side distal of the proximal end of the expansion body, whereby the suspended line portion of the wiring portion extending from the retaining portion to the recess is located at a position away from the linear bodies constituting the expansion body. Accordingly, the medical device can inhibit the linear bodies from interfering with the wiring portion, for example, from catching the wiring portion therebetween, when the radially expanded expansion body is contracted.

In the medical device, the distal extension portion may include a distal tube body that is hollow and that includes an inner peripheral surface of the distal extension portion, and a distal covering portion that is tubular, the distal covering portion covering an outer surface of the distal tube body and functioning as the retaining portion. With this configuration, the distal covering portion can bundle and retain the wiring portion so as not to be separated from the outer surface of the distal extension portion.

In the medical device, at least a part of the linear bodies located in the recess may include an insertion hole, and the wiring portion may penetrate the insertion hole from an inside of the expansion body on a side of the first end with respect to the suspended line portion to expose the first end to the outside of the expansion body. With this configuration, in the medical device, the wiring portion extending at the position away from the linear bodies of the expansion body can be exposed on the surface facing the outside of the recess, and the electrode unit can be appropriately disposed.

In the medical device, the insertion hole may be provided closer to the proximal-side top portion than the electrode unit disposed at the proximal-side upright portion in the recess. With this configuration, in the medical device, the distal part of the wiring portion can be stably placed in the recess.

In the medical device, the insertion hole may be provided closer to the bottom than the electrode unit disposed at the proximal-side upright portion in the recess. With this configuration, in the medical device, the wiring portion extending from the retaining portion to the recess can be positioned on the inner side of the expansion body, whereby it is possible to further inhibit the linear bodies of the contracting expansion body from interfering with the wiring portion.

In the medical device, the insertion hole may be provided closer to the bottom than the electrode unit disposed at the distal-side upright portion in the recess. With this configuration, in the medical device, the wiring portion extending from the retaining portion to the recess can be positioned on the inner side of the expansion body, whereby it is possible to further inhibit the linear bodies of the contracting expansion body from interfering with the wiring portion.

In the medical device, the plurality of linear bodies may branch or join at least once or more from the proximal part toward the recess in the expansion body, and at least a part of the suspended line portion of the wiring portion may be located between linear bodies adjacent to each other in the circumferential direction among the plurality of linear bodies. With this configuration, in the medical device, the wiring portion that is likely to be caught between the linear bodies of the expansion body in terms of the arrangement relationship in the circumferential direction is disposed so as to pass through the inside of the expansion body by the retaining portion, whereby it is possible to inhibit the linear bodies from interfering with the wiring portion.

In the medical device, the shaft portion may include a tube body including the distal tube body and extending from a distal end to a proximal end of the shaft portion, and a covering portion that is tubular, the covering portion including the distal covering portion and covering the tube body, and the connection portion of the shaft portion may be provided on the covering portion adjacent to a proximal end of the distal covering portion. With this configuration, the medical device can be provided with the distal covering portion that retains the wiring portion using the covering portion that covers the shaft portion.

In the medical device, a length of the suspended line portion along a longitudinal direction of the wiring portion may be longer than a linear distance between the insertion hole and a section where the suspended line portion is exposed from the retaining portion to the interior of the expansion body. With this configuration, in the medical device, the wiring portion is less likely to receive a load during expansion of the expansion body, whereby breakage of the wiring portion can be inhibited.

### BRIEF DESCRIPTION OF THE DRAWINGS

FIG. 1 is a front view illustrating an overall configuration of a medical device according to an embodiment;

FIG. 2 is an enlarged perspective view of the vicinity of an expansion body;

FIG. 3 is an enlarged front view of the vicinity of the expansion body;

FIG. 4 is an enlarged front view of a shaft and the expansion body that is briefly illustrated;

FIG. 5 is an enlarged cross-sectional view of the shaft and the expansion body that is briefly illustrated;

FIG. 6 is an enlarged perspective view illustrating the vicinity of an electrode unit of the expansion body;

FIG. 7 is an enlarged cross-sectional view of a shaft including a bundling portion and an expansion body that is briefly illustrated according to a modification;

FIG. 8 is an enlarged perspective view of the vicinity of an electrode unit of an expansion body including the electrode unit according to a modification;

FIG. 9 is an enlarged cross-sectional view of a shaft and an expansion body briefly illustrated and including the electrode unit in FIG. 8;

FIG. 10 is an enlarged cross-sectional view of a shaft and an expansion body when an electrode unit is inserted into the expansion body from the side closer to a bottom;

FIG. 11 is an enlarged cross-sectional view of a shaft and an expansion body in a case where an electrode unit is disposed on a distal-side upright portion;

FIG. 12 is an explanatory diagram schematically illustrating a state where the expansion body is placed in an atrial septum, in which the medical device is illustrated as a front view and a biological tissue is illustrated as a cross-sectional view; and

FIG. 13 is an enlarged front view illustrating a state in which the expansion body grips the biological tissue.

### DETAILED DESCRIPTION

Hereinafter, an embodiment of the medical device having an expansion body, representing examples of the new medical device having an expansion body disclosed here will be described with reference to the drawings. Note that dimensional ratios in the drawings may be exaggerated and different from actual ratios for convenience of description. In addition, in the present specification, a side of a medical device 10 that is to be inserted into a biological lumen will be referred to as a “distal end” or a “distal side”, and a side near the operator's hand operating the medical device 10 will be referred to as a “proximal end” or a “proximal side”.

The medical device according to the embodiment described below expands a first through hole Hh1 formed in an atrial septum HA of the heart H of a patient to form a second through hole Hh2, i.e., to transform first, unexpanded through hole Hh1 into second, expanded through hole Hh2, and to further perform a maintenance procedure to maintain the expanded second through hole Hh2 at the increased size to obtain a communication hole Hh, i.e., to transform second through hole Hh2 into communication hole Hh by performing the maintenance procedure.

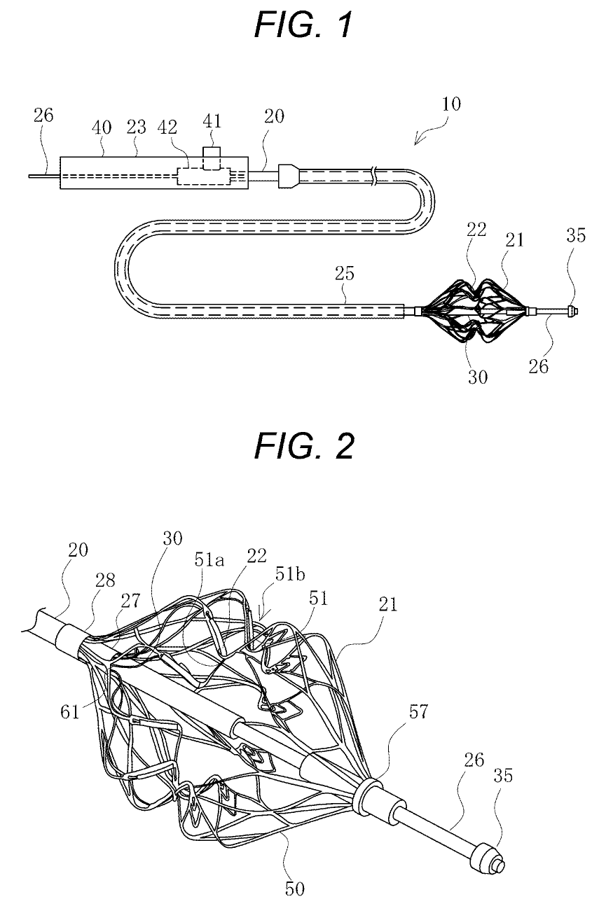

As illustrated in FIG. 1, the medical device 10 according to the present embodiment includes an elongated hollow shaft portion 20, an expansion body 21 disposed on a distal part of the shaft portion 20, and a manual operation unit 23 disposed on a proximal part of the shaft portion 20. The expansion body 21 has an electrode unit 22 which is an energy transfer element for performing the above-described maintenance procedure.

The shaft portion 20 includes, at the distal part, a hollow distal extension portion 30 extending to the interior from a proximal end of the expansion body 21. The distal extension portion 30 extends along a central axis of the expansion body 21 from the vicinity of the proximal end of the expansion body 21 to the middle of the expansion body 21, specifically, to the vicinity of a recess 51 of the expansion body 21. In addition, the shaft portion 20 has a connection portion 28 connected to the proximal part of the expansion body 21.

The shaft portion 20 includes a storage sheath 25 disposed on an outer peripheral portion. The shaft portion 20 is movable forward and rearward in an axial direction with respect to the storage sheath 25. The storage sheath 25 can store the expansion body 21 therein when moved to the distal side of the shaft portion 20. The expansion body 21 can be exposed from the storage sheath 25 by moving the storage sheath 25 that has stored the expansion body 21 to the proximal side.

A pulling shaft 26 is disposed in the shaft portion 20 so as to be slidable with respect to the shaft portion 20. The pulling shaft 26 is disposed from a position proximal of the manual operation unit 23 to a position distal of the expansion body 21. The pulling shaft 26 protrudes from the distal part of the shaft portion 20, specifically, from the distal extension portion 30, passes through the inside of the expansion body 21, and protrudes from the distal end of the expansion body 21. The distal part of the pulling shaft 26 is fixed to the distal end member 35.

The distal end member 35 to which the distal part of the pulling shaft 26 is fixed is not fixed to the expansion body 21. As a result, when the pulling shaft 26 slides in a proximal direction with respect to the shaft portion 20, the distal end member 35 can apply a compressive force to the expansion body 21 along the axial center of the shaft portion 20. In addition, when the expansion body 21 is stored in the storage sheath 25, the distal end member 35 is moved away from the expansion body 21 to the distal side, by which the expansion body 21 can be rather easily moved in a direction of extension of the expansion body 21, and thus, storage capability can be improved.

The manual operation unit 23 has a housing 40 to be held by an operator, an operation dial 41 that can be rotationally operated by the operator, and a conversion mechanism 42 operated in conjunction with the rotation of the operation dial 41. The pulling shaft 26 is held by the conversion mechanism 42 inside the manual operation unit 23. The conversion mechanism 42 can move the held pulling shaft 26 forward and rearward in the axial direction in conjunction with the rotation of the operation dial 41. For example, a rack and pinion mechanism can be used as the conversion mechanism 42.

It is preferable that the shaft portion 20 is formed of a material having a certain degree of flexibility. Examples of such a material include polyolefin such as polyethylene, polypropylene, polybutene, ethylene-propylene copolymer, ethylene-vinyl acetate copolymer, ionomer, or a mixture of two or more of them, soft polyvinyl chloride resin, polyamide, polyamide elastomer, polyester, polyester elastomer, polyurethane, fluorine resin such as polytetrafluoroethylene, polyimide, PEEK, silicone rubber, and latex rubber.

The pulling shaft 26 can be formed of, for example, an elongated linear body including a super elasticity alloy such as a nickel-titanium alloy and a copper-zinc alloy, a metal material such as stainless steel, a resin material having comparatively high rigidity, or the like.

The distal end member 35 can be formed of, for example, a super elasticity alloy such as a nickel-titanium alloy or a copper-zinc alloy, a metal material such as stainless steel, a polymer material such as polyolefin, polyvinyl chloride, polyamide, polyamide elastomer, polyurethane, polyurethane elastomer, polyimide, or fluorine resin, or a mixture of the above polymer materials. Alternatively, the distal end member 35 can be formed of a multilayer tube containing two or more kinds of polymer materials.

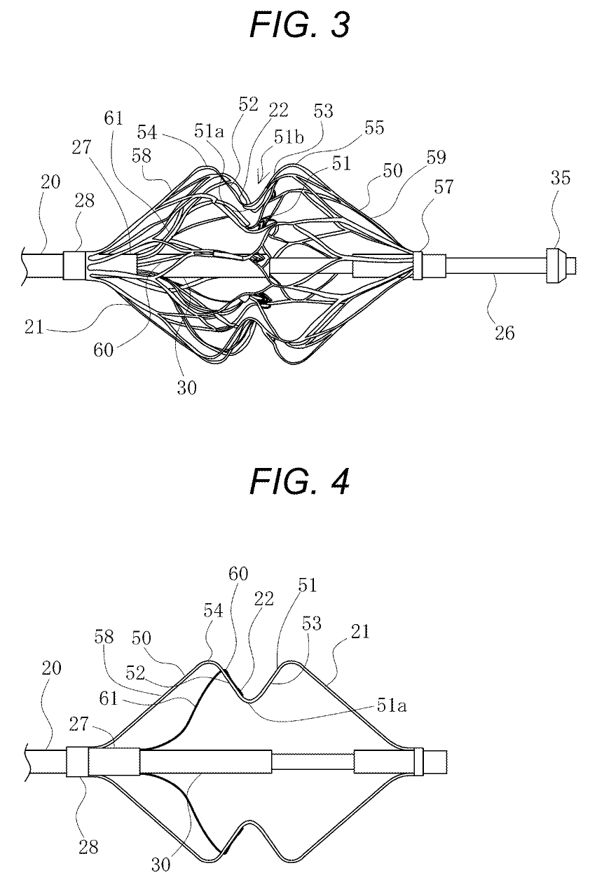

As illustrated in FIGS. 2 and 3, the expansion body 21 includes a plurality of linear bodies 50 in a circumferential direction. The linear bodies 50 form a mesh-shaped structure by branching and joining along a length direction between the connection portion 28 to which the proximal ends are connected and a distal convergence portion 57 to which the distal ends are connected. As a result, the expansion body 21 can expand and contract in a radial direction. The distance between the linear bodies 50 adjacent to each other in the circumferential direction among the plurality of linear bodies 50 constituting the expansion body 21 changes according to the expansion and contraction of the expansion body 21. The proximal parts of the linear bodies 50 extend to the distal side from the connection portion 28 of the expansion body 21. The distal parts of the linear bodies 50 extend to the proximal side from the distal convergence portion 57 of the expansion body 21. In a state where the expansion body 21 is expanded, the linear bodies 50 have a proximal-side inclined portion 58 that is increased in diameter toward a proximal-side top portion 54 from the connection portion 28 at the proximal end. In addition, the linear bodies 50 have a distal-side inclined portion 59 that is increased in diameter from the distal convergence portion 57 toward a distal-side top portion 55. The linear bodies 50 have a recess 51 recessed inward in the radial direction of the expansion body 21 at the central part in the axial direction. An innermost part of the recess 51 in the radial direction is a bottom 51a. The recess 51 defines a reception space 51b that can receive a biological tissue when the expansion body 21 is expanded.

The recess 51 includes a proximal-side upright portion 52 extending radially outward from the proximal end of the bottom 51a to the proximal-side top portion 54, and a distal-side upright portion 53 extending radially outward from the distal end of the bottom 51a. When the pulling shaft 26 slides in the proximal direction with respect to the shaft portion 20 to apply a compressive force to the expansion body 21, the distal-side upright portion 53 and the proximal-side upright portion 52 are brought close to each other, and both portions come in close contact with the biological tissue received in the reception space 51b. The electrode unit 22 is disposed along the recess 51 at the proximal-side upright portion 52 so as to face the reception space 51b. That is, the electrode unit 22 is provided along the expansion body 21 at an intermediate part of the expansion body 21 in the central axis direction so as to be exposed to the outside of the expansion body 21. In the present embodiment, ten electrode units 22 are disposed along the circumferential direction. The electrode unit 22 may be disposed on the distal-side upright portion 53.

The linear bodies 50 constituting the expansion body 21 can be formed by, for example, cutting a single metal cylindrical member with laser. The linear bodies 50 can be formed of a metal material. Examples of the metal material that may be used include a titanium-based (Ti-Ni, Ti-Pd, Ti-Nb-Sn, etc.) alloy, a copper-based alloy, stainless steel, β-titanium steel, and a Co-Cr alloy. It is more preferable to use an alloy having spring property such as a nickel titanium alloy. However, the material of the linear bodies 50 is not limited to the above materials, and other materials may be used.

The electrode units 22 are connected to an energy supply device (not illustrated) which is an external device. A high-frequency voltage is applied from the energy supply device to an electrode pair including two electrode units 22, and energy is applied between them. In other words, in the embodiment, the electrode unit 22 is configured as a bipolar electrode. The electrode unit 22 may also be a monopolar electrode. In this case, electric current is supplied between the electrode unit 22 and an external electrode.

An electrode assembly 60 that is a separate component from the expansion body 21 is attached to the expansion body 21. As illustrated in FIG. 3, the electrode assembly 60 includes an elongated wiring portion 61 and an electrode unit 22 provided at a distal part of the wiring portion 61. A conductive surface of the electrode unit 22 is exposed. The surface of the wiring portion 61 is covered with an insulating layer.

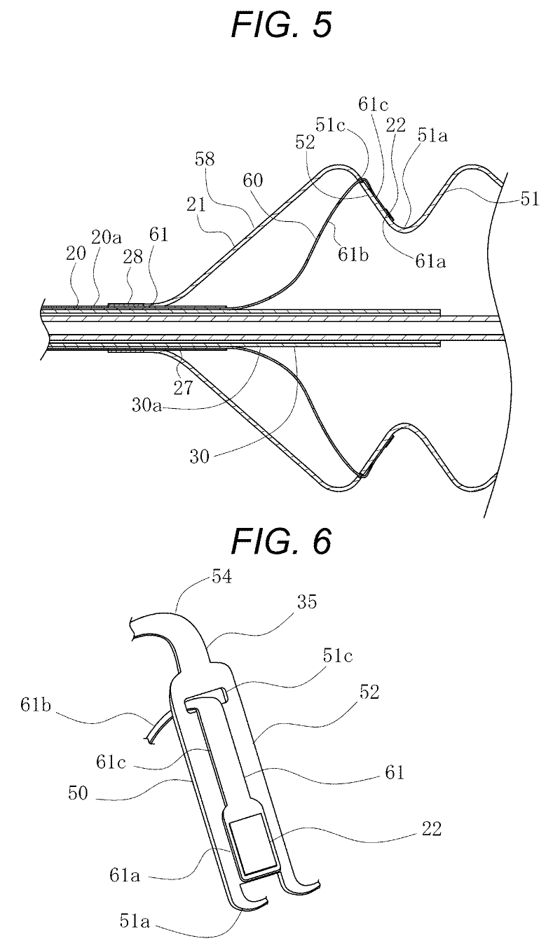

As illustrated in FIGS. 4 and 5, the wiring portion 61 constituting the electrode assembly 60 includes a first end 61a connected to the electrode unit 22, a second end (not illustrated) opposite to the first end 61a, and a suspended line portion 61b disposed between the first end 61a and the second end. In the wiring portion 61, a section on the second end side extends along the shaft portion 20, a section of the suspended line portion 61b extends away from the shaft portion 20 toward the recess 51 of the linear bodies 50 within the expansion body 21 and penetrates the linear bodies 50, and the first end 61a provided with the electrode unit 22 is exposed and extends to the outside of the expansion body 21. A tubular distal covering portion 27 that bundles and retains a plurality of wiring portions 61 included in a plurality of electrode assemblies 60 on the outer surface of the distal extension portion 30 is provided between the connection portion 28 of the expansion body 21 and the distal end of the distal extension portion 30. Therefore, from the proximal side to the distal side, the wiring portion 61 extends along the shaft portion 20 to the distal end position of the distal covering portion 27, and is separated from the shaft portion 20 on the side distal of the distal end position of the distal covering portion 27. The distal covering portion 27 can be formed of a resin material same as that of the shaft portion 20. As described above, the distal extension portion 30 includes, between the connection portion 28 of the shaft portion 20 and the distal end of the distal extension portion 30, the distal covering portion 27 that functions as a retaining portion for retaining a section of the wiring portion 61 proximal of the suspended line portion 61b to the distal extension portion 30 on the outer side of the inner peripheral surface of the distal extension portion 30.

As illustrated in FIG. 5, the shaft portion 20 on the side proximal of the expansion body 21 has a covering portion 20a that covers the outer surface, and the wiring portions 61 of the electrode assemblies 60 extend between the covering portion 20a and the outer surface of the shaft portion 20. The distal extension portion 30 has a hollow distal tube body 30a including an inner peripheral surface of the distal extension portion 30 and the tubular distal covering portion 27 covering an outer surface of the distal tube body 30a. The distal covering portion 27 extends from the inside of the connection portion 28 of the expansion body 21 to the interior of the expansion body 21. The wiring portions 61 extend between the distal covering portion 27 and the outer surface of the distal tube body 30a. Thus, the wiring portions 61 are bundled on the outer surface of the distal extension portion 30 located within the expansion body 21. The positions where the wiring portions 61 are separated from the shaft portion 20 are located distal of the connection portion 28 of the expansion body 21 because the wiring portions 61 are bundled by means of the distal covering portion 27 within the expansion body 21 on the side distal of the connection portion 28. That is, the plurality of wiring portions 61 is retained within the expansion body 21 on the side distal of the proximal end of the expansion body 21 by the distal covering portion 27 serving as the retaining portion. Therefore, the suspended line portions 61b of the wiring portions 61 are aerially disposed to extend toward the recess 51 at positions away inward from the proximal-side inclined portion 58 of the linear bodies 50 within the expansion body 21. That is, the suspended line portions 61b of the wiring portions 61 extending from the location (in the present embodiment, the distal end position of the retaining portion) of the distal covering portion 27 (retaining portion) where the suspended line portions 61b are exposed to the recess 51 are disposed at positions away from the linear bodies 50 constituting the expansion body 21. Therefore, the suspended line portions 61b are retained neither on the expansion body 21 nor on the distal extension portion 30 in the expanded state of the expansion body 21. Thus, when the radially expanded expansion body 21 is drawn into and stored in the storage sheath 25, the wiring portions 61 are less likely to come into contact with the proximal-side inclined portion 58 of the linear bodies 50 that are being contracted, whereby it is possible to inhibit the wiring portions 61 from being damaged due to the interference with the linear bodies 50. Since the expansion body 21 has a shape in which the linear bodies 50 branch and join, at least a part of the suspended line portion 61b of each of the wiring portions 61 along the length direction is disposed between the linear body 50 and the linear body 50 adjacent to each other in the circumferential direction. From this structure, the wiring portions 61 may be caught and damaged between the linear body 50 and the linear body 50 adjacent to each other in the circumferential direction at the time of contraction of the expansion body 21, but since the wiring portions 61 are disposed on the inner side away from the linear bodies 50 by the distal covering portion 27, the possibility of being caught between the linear bodies 50 can be reduced.

As illustrated in FIG. 6, the linear bodies 50 have an insertion hole 51c in the proximal-side upright portion 52. The insertion hole 51c is an opening that does not substantially change in shape or size in both the expanded state and the contracted state of the expansion body 21. The wiring portion 61 is inserted into the insertion hole 51c, by which the distal part including the electrode unit 22 is exposed to the outside of the expansion body 21. The wiring portion 61 includes an exposed portion 61c exposed to the outside of the expansion body 21 between the suspended line portion 61b and the first end 61a. The insertion hole 51c is provided closer to the proximal-side top portion 54 than the electrode unit 22 disposed near the bottom 51a in the proximal-side upright portion 52 of the recess 51. Therefore, the wiring portion 61 passing through the insertion hole 51c extends toward the bottom 51a of the recess 51. The length of the suspended line portion 61b along the longitudinal direction of the wiring portion 61 is longer than the linear distance between the insertion hole 51c and the location where the suspended line portion 61b is exposed from the distal covering portion 27 to the interior of the expansion body 21 (see FIG. 5). Therefore, when the expansion body 21 is expanded, the wiring portions 61 are less likely to receive a load, so that breakage of the wiring portions 61 can be inhibited.

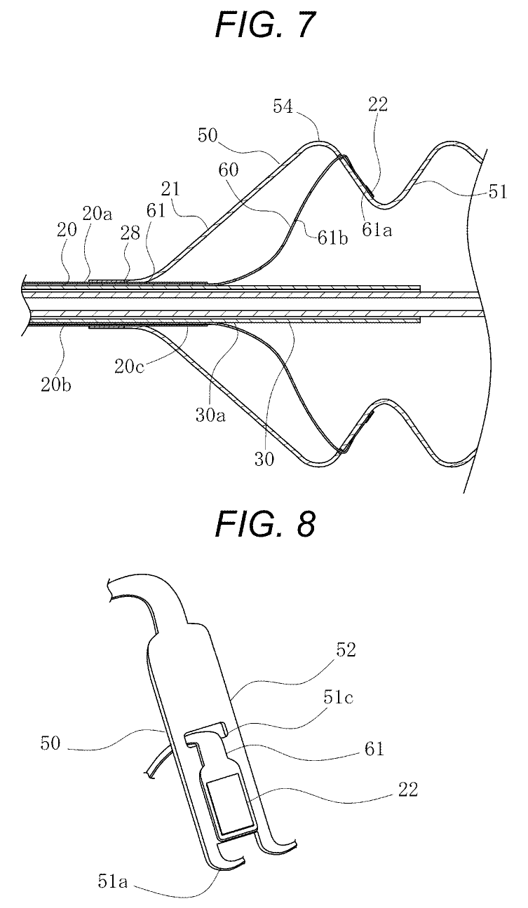

As illustrated in FIG. 7, the distal part of the covering portion 20a that constitutes the shaft portion 20 and covers a tube body 20b including the distal tube body 30a may be used as the distal covering portion 20c. In this case, the covering portion 20a is extended from the side proximal of the expansion body 21 to the interior of the expansion body 21 beyond the connection portion 28 of the expansion body 21, and the distal covering portion 20c retains the wiring portions 61. The wiring portion 61 of the electrode assembly 60 extends along the shaft portion 20 in the region where the covering portion 20a is provided, and the suspended line portion 61b extending away from the shaft portion 20 toward the recess 51 and the first end 61a exposed to the outside of the expansion body 21 and connected to the electrode unit 22 are located distal of the distal end position of the covering portion 20a.

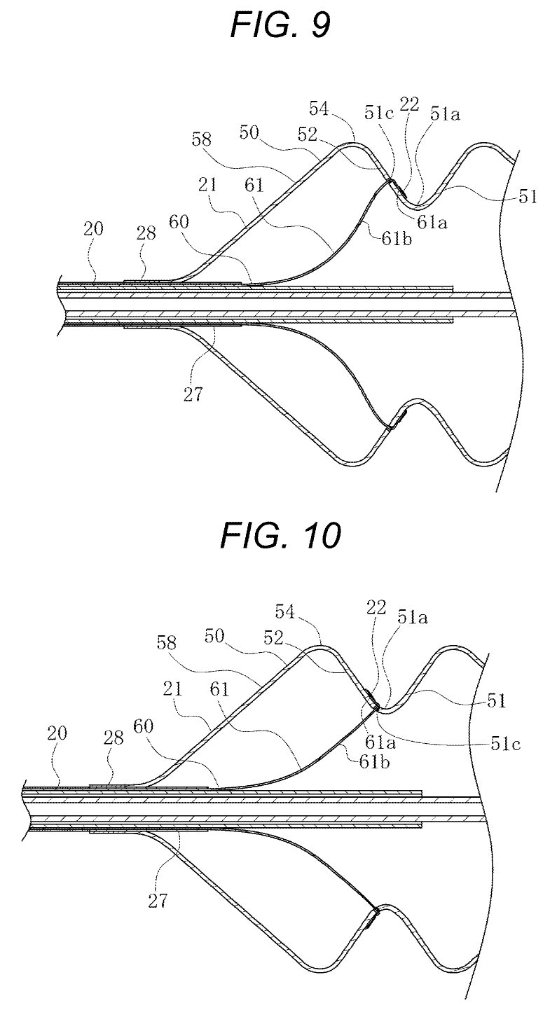

As illustrated in FIG. 8, the insertion hole 51c formed in the linear bodies 50 may be formed at a position close to the bottom 51a of the recess 51. In this case, as illustrated in FIG. 9, the radial positions of the distal covering portion 27 and the insertion hole 51c are close to each other, and thus, the suspended line portion 61b of the wiring portion 61 of the electrode assembly 60 passes through the region further inside the proximal-side inclined portion 58 of the linear bodies 50, and the wiring portion 61 penetrates the linear bodies 50 at a position far away from the proximal-side top portion 54. Therefore, the possibility of interference between the wiring portion 61 and the linear bodies 50 during contraction of the expansion body 21 can be further reduced.

As illustrated in FIG. 10, the insertion hole 51c may be formed at a position of the proximal-side upright portion 52 close to the bottom 51a, and the first end 61a of the wiring portion 61 penetrating the insertion hole 51c may extend from the bottom 51a toward the proximal-side top portion 54. That is, the insertion hole 51c may be provided closer to the bottom 51a than the electrode unit 22. With this configuration, the wiring portion 61 of the electrode assembly 60 penetrates the linear bodies 50 at a position farther from the proximal-side top portion 54, whereby the suspended line portion 61b of the wiring portion 61 extending from the distal covering portion 27 to the recess 51 is located more inward, and the possibility of interference of the wiring portions 61 with the linear bodies 50 during contraction of the expansion body 21 can be further reduced.

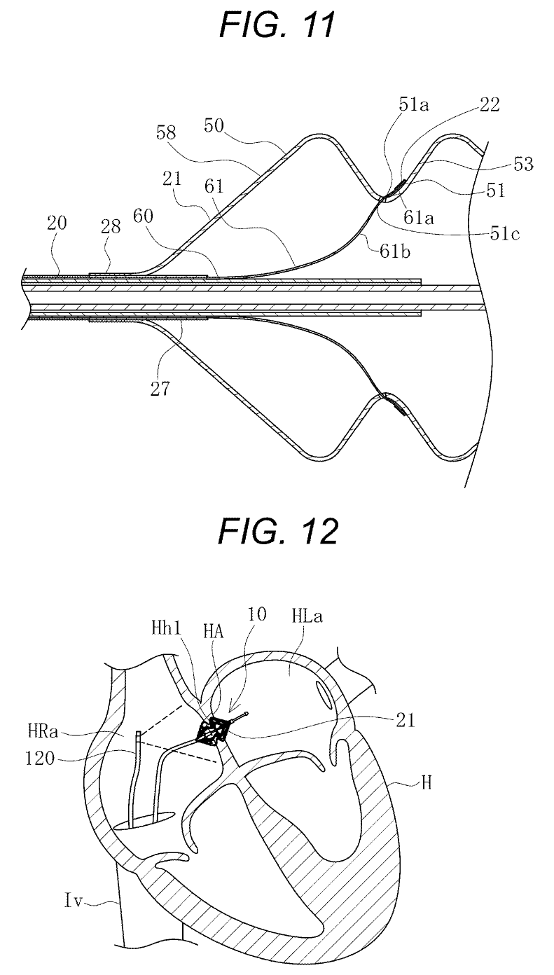

As illustrated in FIG. 11, the electrode unit 22 may be disposed on the distal-side upright portion 53 of the recess 51. In this case, the insertion hole 51c is provided closer to the bottom 51a than the electrode unit 22 disposed on the distal-side upright portion 53 in the recess 51. By disposing the electrode unit 22 on the distal-side upright portion 53, there is no section where the wiring portion 61 extending from the distal covering portion 27 to the electrode unit 22 is folded back, so that the suspended line portion 61b of the wiring portion 61 can be disposed more inward, whereby the possibility of interference of the wiring portions 61 with the linear bodies 50 during contraction of the expansion body 21 can be further reduced.

When the medical device 10 is used, the first through hole Hh1 is formed in advance at the position of the fossa ovalis of the atrial septum HA. The medical device 10 expands and increases in width the first through hole Hh1 to form a substantially circular second through hole Hh2, and cauterizes the edge of the second through hole Hh2. As a result, the medical device 10 forms the communication hole Hh of which size, that is, patency is maintained. As illustrated in FIG. 12, the medical device 10 is delivered from an inferior vena cava Iv to the vicinity of the atrial septum HA via a right atrium HRa, and the expansion body 21 is placed at the position of the first through hole Hh1 formed in advance. The distal part of the medical device 10 penetrates the atrial septum HA and reaches the left atrium HLa.

During the insertion of the medical device 10, the expansion body 21 is stored and contracted in the storage sheath 25, and after the storage sheath 25 penetrates the atrial septum HA, the storage sheath 25 is moved to the proximal side, by which the expansion body 21 can be exposed. When the expansion body 21 is exposed, the expansion body 21 radially expands, and the recess 51 is placed in the first through hole Hh1 of the atrial septum HA and receives a biological tissue surrounding the first through hole Hh1 in the reception space 51b.

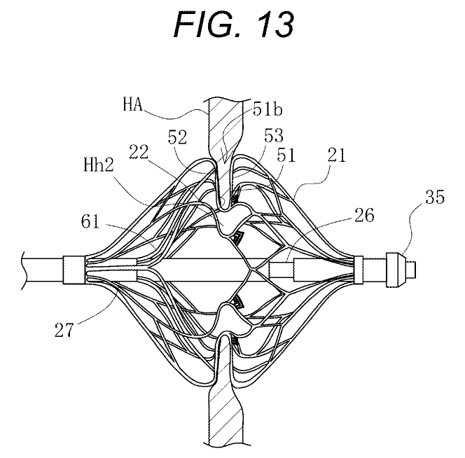

The pulling shaft 26 is moved to the proximal side in a state in which the reception space 51b receives the biological tissue, by which the expansion body 21 is pulled in a compression direction by the distal end member 35 to be compressed in the axial direction, the atrial septum HA is gripped by the proximal-side upright portion 52 and the distal-side upright portion 53 which constitute the recess 51, and the electrode units 22 are pressed against the biological tissue, as illustrated in FIG. 13. At this time, as the radial position of the recess 51 moves outward, the first through hole Hh1 is expanded in the radial direction, and the second through hole Hh2 is formed. The diameter of the second through hole Hh2 is larger than that of the first through hole Hh1. The fossa ovalis where the second through hole Hh2 is formed has a smaller wall thickness than other parts of the atrial septum HA. Therefore, the recess 51 of the expansion body 21 can hold the biological tissue surrounding the periphery of the second through hole Hh2 so as to press the electrode units 22 against the biological tissue.

In a state where the electrode units 22 are pressed against the biological tissue, high frequency energy is applied to the edge of the second through hole Hh2, that is, the biological tissue surrounding the second through hole Hh2, through the electrode units 22, whereby the edge of the second through hole Hh2 can be cauterized (heated and cauterized) by the high frequency energy. The high frequency energy is applied by applying a voltage between the pair of electrode units 22 adjacent to each other in the circumferential direction. This results in making it possible to inhibit blockage due to natural healing of the communication hole Hh and maintain a size thereof. When the expansion body 21 is contracted and removed after the cauterization, the second through hole Hh2 is slightly contracted in the radial direction, and the communication hole Hh is formed.

When the medical device 10 is used, hemodynamics is checked by a hemodynamics checking device 120 delivered to the right atrium HRa via the inferior vena cava Iv. As the hemodynamics checking device 120, a known echo catheter can be used, for example. The operator can display an echo image obtained by the hemodynamics checking device 120 on a display device, such as a display, and can check the volume of blood passing through the communication hole Hh on the basis of a displayed result.

When the expansion body 21 is removed after the cauterization by the electrode units 22, the storage sheath 25 is moved forward with respect to the shaft portion 20, and the expansion body 21 is drawn into the storage sheath 25 from the proximal side. At this time, the wiring portions 61 located at positions away inward from the linear bodies 50 of the expansion body 21 by the distal covering portion 27 are less likely to interfere with the contracting linear bodies 50, and thus, the possibility of breakage of the wiring portions 61 by the linear bodies 50 can be reduced.

As described above, (1) a medical device 10 according to the present embodiment includes: an expansion body 21 including a plurality of linear bodies 50 and expandable and contractible in a radial direction; a shaft portion 20 that includes a connection portion 28 connected to a proximal part of the expansion body 21 and that is elongated; and at least one electrode assembly 60 including an electrode unit 22 and a wiring portion 61 that is elongated and that is connected to the electrode unit 22, wherein the expansion body 21 includes a recess 51 recessed radially inward when the expansion body 21 is expanded, and is formed in such a manner that a distance between linear bodies 50 adjacent to each other in a circumferential direction among the plurality of linear bodies 50 constituting the expansion body 21 changes according to expansion and contraction of the expansion body 21, the recess 51 includes a bottom 51a located on an innermost side in the radial direction, a proximal-side upright portion 52 extending from a proximal end of the bottom 51a toward a proximal-side top portion 54 on a radially outer side, and a distal-side upright portion 53 extending from a distal end of the bottom 51a toward a distal-side top portion 55 on a radially outer side, the electrode unit 22 is disposed along the proximal-side upright portion 52 or the distal-side upright portion 53 so as to be exposed to an outside of the expansion body 21, the shaft portion 20 includes a distal extension portion 30 that is hollow and that extends from the connection portion 28 to a distal side within the expansion body 21, the wiring portion 61 of the at least one electrode assembly 60 includes a first end 61a connected to the electrode unit 22, a second end on an opposite side of the first end 61a, and a suspended line portion 61b disposed between the first end 61a and the second end, the distal extension portion 30 includes a retaining portion 27 between the connection portion 28 of the shaft portion 20 and a distal end of the distal extension portion 30, the retaining portion 27 retaining a section of the wiring portion 61 on a side proximal of the suspended line portion 61b on the distal extension portion 30 on an outside of an inner peripheral surface of the distal extension portion 30, and the suspended line portion 61b of the wiring portion 61 extends from the retaining portion 27 of the distal extension portion 30 to the proximal-side upright portion 52 or the distal-side upright portion 53 through an interior of the expansion body 21, and is aerially positioned within the expansion body 21 in an expanded state of the expansion body 21. In the medical device 10 configured as described above, a plurality of the wiring portions 61 is retained by the retaining portion 27 in the interior of the expansion body 21 on the side distal of the proximal end of the expansion body 21, whereby the suspended line portions 61b of the wiring portions 61 extending from the retaining portion 27 to the recess 51 are located at positions away from the linear bodies 50 constituting the expansion body 21. Accordingly, the medical device 10 can inhibit the linear bodies 50 from interfering with the wiring portion 61, for example, from catching the wiring portion 61 therebetween, when the radially expanded expansion body 21 is contracted.

(2) In the medical device 10 according to (1), the distal extension portion 30 may include a distal tube body 30a that is hollow and that includes an inner peripheral surface of the distal extension portion 30, and a distal covering portion 27 that is tubular, the distal covering portion 27 covering an outer surface of the distal tube body 30a and functioning as the retaining portion. With this configuration, the distal covering portion 27 can bundle and retain the wiring portions 61 so as not to be separated from the outer surface of the distal extension portion 30.

(3) In the medical device 10 according to (1) or (2), at least a part of the linear bodies 50 located in the recess 51 may include an insertion hole 51c, and the wiring portion 61 may penetrate the insertion hole 51c from an inside of the expansion body 21 on a side of the first end 61a with respect to the suspended line portion 61b to expose the first end 61a to the outside of the expansion body 21. With this configuration, in the medical device 10, the wiring portion 61 extending at the position away from the linear bodies 50 of the expansion body 21 can be exposed on the surface facing the outside of the recess 51, and the electrode unit 22 can be appropriately disposed.

(4) In the medical device 10 according to (3), the insertion hole 51c may be provided closer to the proximal-side top portion 54 than the electrode unit 22 disposed at the proximal-side upright portion 52 in the recess 51. With this configuration, in the medical device 10, the distal part of the wiring portion 61 can be stably placed in the recess 51.

(5) In the medical device 10 according to (3), the insertion hole 51c may be provided closer to the bottom 51a than the electrode unit 22 disposed at the proximal-side upright portion 52 in the recess 51. With this configuration, in the medical device 10, the wiring portion 61 extending from the retaining portion 27 to the recess 51 can be positioned on the inner side of the expansion body 21, whereby it is possible to further inhibit the linear bodies 50 of the contracting expansion body 21 from interfering with the wiring portion 61.

(6) In the medical device 10 according to (3), the insertion hole 51c may be provided closer to the bottom 51a than the electrode unit 22 disposed at the distal-side upright portion 53 in the recess 51. With this configuration, in the medical device 10, the wiring portion 61 extending from the retaining portion 27 to the recess 51 can be positioned on the inner side of the expansion body 21, whereby it is possible to further inhibit the linear bodies 50 of the contracting expansion body 21 from interfering with the wiring portion 61.

(7) In the medical device 10 according to any one of (1) to (6), the plurality of linear bodies 50 may branch or join at least once or more from the proximal part toward the recess 51 in the expansion body 21, and at least a part of the suspended line portion 61b of the wiring portion 61 may be located between linear bodies 50 adjacent to each other in the circumferential direction among the plurality of linear bodies 50. With this configuration, in the medical device 10, the wiring portion 61 that is likely to be caught between the linear bodies 50 of the expansion body 21 in terms of the arrangement relationship in the circumferential direction is disposed so as to pass through the inside of the expansion body 21 by the retaining portion 27, whereby it is possible to inhibit the linear bodies 50 from interfering with the wiring portion 61.

(8) In the medical device 10 according to (2), the shaft portion 20 may include a tube body 20b including the distal tube body 30a and extending from a distal end to a proximal end of the shaft portion 20, and a covering portion 20a that is tubular, the covering portion 20a including the distal covering portion 20c and covering the tube body 20b, and the connection portion 28 of the shaft portion 20 is provided on the covering portion 20a adjacent to a proximal end of the distal covering portion 20c. With this configuration, the medical device 10 can be provided with the distal covering portion 20c that retains the wiring portion 61 using the covering portion 20a that covers the shaft portion 20.

(9) In the medical device 10 according to any one of (3) to (6), a length of the suspended line portion 61b along a longitudinal direction of the wiring portion 61 may be longer than a linear distance between the insertion hole 51c and a section where the suspended line portion 61b is exposed from the retaining portion 27 to the interior of the expansion body 21. With this configuration, in the medical device 10, the wiring portion 61 is less likely to receive a load during expansion of the expansion body 21, whereby breakage of the wiring portion 61 can be inhibited.

The detailed description above describes embodiments of a medical device having an expansion body representing examples of the new medical device having an expansion body disclosed here. The invention is not limited, however, to the precise embodiment and modifications described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims. For example, in the above-described embodiments, the retaining portion that retains and bundles the sections of the wiring portions 61 proximal of the suspended line portions 61b with the distal extension portion 30 is the tubular distal covering portion 27, but the retaining portion only needs to be able to retain the wiring portions 61 outside the inner peripheral surface of the distal extension portion 30, and may have a configuration other than the tubular member. For example, a part of the shaft portion 20 including the distal extension portion 30 may be a two-layer tube having an inner layer and an outer layer on the outer side of the inner layer, and the wiring portions 61 may be held between the inner layer and the outer layer to be retained within an area of the distal extension portion 30 in the major axis direction. Alternatively, a part of the shaft portion 20 including the distal extension portion 30 may be formed as a multi-lumen tube having a plurality of lumens, and the wiring portions 61 may be stored in a lumen formed outside a lumen at a central part through which the pulling shaft 26 is inserted, by which the wiring portions 61 are retained within an area of the distal extension portion 30 in the major axis direction. Alternatively, the wiring portions 61 may be fixed to the outer peripheral surface of the distal extension portion 30 with an adhesive, and the wiring portions 61 may extend from the distal end of the section fixed with the adhesive toward the recess 51 of the linear bodies 50..

What is claimed is:

1. A medical device comprising:
an expansion body including a plurality of linear bodies and expandable and contractible in a radial direction;
a shaft portion that includes a connection portion connected to a proximal part of the expansion body and that is elongated and hollow; and
at least one electrode assembly including an electrode unit and a wiring portion that is elongated and that is connected to the electrode unit, wherein
the expansion body includes a recess recessed radially inward when the expansion body is expanded, and is formed in such a manner that a distance between linear bodies adjacent to each other in a circumferential direction among the plurality of linear bodies constituting the expansion body changes according to expansion and contraction of the expansion body,
the recess includes a bottom located on an innermost side in the radial direction, a proximal-side upright portion extending from a proximal end of the bottom toward a proximal-side top portion on a radially outer side, and a distal-side upright portion extending from a distal end of the bottom toward a distal-side top portion on a radially outer side,
the electrode unit is disposed along the proximal-side upright portion or the distal-side upright portion so as to be exposed to an outside of the expansion body,
the shaft portion includes a distal extension portion that is hollow and that extends from the connection portion to a distal side within the expansion body,
the wiring portion of the at least one electrode assembly includes a first end connected to the electrode unit, a second end on an opposite side of the first end, and a suspended line portion disposed between the first end and the second end,
the distal extension portion includes a retaining portion between the connection portion of the shaft portion and a distal end of the distal extension portion, the retaining portion retaining a section of the wiring portion on a side proximal of the suspended line portion on the distal extension portion on an outside of an inner peripheral surface of the distal extension portion, and
the suspended line portion of the wiring portion extends from the retaining portion of the distal extension portion to the proximal-side upright portion or the distal-side upright portion through an interior of the expansion body, and is aerially positioned within the expansion body in an expanded state of the expansion body.

2. The medical device according to claim 1, wherein the distal extension portion includes a distal tube body that is hollow and that includes an inner peripheral surface of the distal extension portion, and a distal covering portion that is tubular, the distal covering portion covering an outer surface of the distal tube body and functioning as the retaining portion.

3. The medical device according to claim 1, wherein at least a part of the linear bodies located in the recess includes an insertion hole, and
the wiring portion penetrates the insertion hole from an inside of the expansion body on a side of the first end with respect to the suspended line portion to expose the first end to the outside of the expansion body.

4. The medical device according to claim 3, wherein the insertion hole is provided closer to the proximal-side top portion than the electrode unit disposed at the proximal-side upright portion in the recess.

5. The medical device according to claim 3, wherein the insertion hole is provided closer to the bottom than the electrode unit disposed at the proximal-side upright portion in the recess.

6. The medical device according to claim 3, wherein the insertion hole is provided closer to the bottom than the electrode unit disposed at the distal-side upright portion in the recess.

7. The medical device according to claim 1, wherein the plurality of linear bodies branches or joins at least once or more from the proximal part toward the recess in the expansion body, and
at least a part of the suspended line portion of the wiring portion is located between linear bodies adjacent to each other in the circumferential direction among the plurality of linear bodies.

8. The medical device according to claim 2, wherein the shaft portion includes a tube body including the distal tube body and extending from a distal end to a proximal end of the shaft portion, and a covering portion that is tubular, the covering portion including the distal covering portion and covering the tube body, and
the connection portion of the shaft portion is provided on the covering portion adjacent to a proximal end of the distal covering portion.

9. The medical device according to claim 3, wherein a length of the suspended line portion along a longitudinal direction of the wiring portion is longer than a linear distance between the insertion hole and a section where the suspended line portion is exposed from the retaining portion to the interior of the expansion body.

10. A medical device comprising:
an expansion body including a plurality of linear bodies and expandable and contractible in a radial direction;
a shaft portion that includes a connection portion connected to a proximal part of the expansion body and that is elongated and hollow; and
at least one electrode assembly including an electrode unit and a wiring portion that is elongated and that is connected to the electrode unit, wherein
the expansion body includes a recess recessed radially inward when the expansion body is expanded, and is formed in such a manner that a distance between linear bodies adjacent to each other in a circumferential direction among the plurality of linear bodies constituting the expansion body changes according to expansion and contraction of the expansion body,
the electrode unit is disposed along the recess so as to be exposed to an outside of the expansion body,
the shaft portion includes a distal extension portion that is hollow and that extends from the connection portion to a distal side within the expansion body,
the wiring portion of the at least one electrode assembly includes a first end connected to the electrode unit, a second end on an opposite side of the first end, and a suspended line portion disposed between the first end and the second end,
the distal extension portion includes a retaining portion between the connection portion of the shaft portion and a distal end of the distal extension portion, the retaining portion retaining a section of the wiring portion on a side proximal of the suspended line portion on the distal extension portion on an outside of an inner peripheral surface of the distal extension portion, and
the suspended line portion of the wiring portion extends from the retaining portion of the distal extension portion to the recess through an interior of the expansion body, and is aerially positioned within the expansion body in an expanded state of the expansion body.

11. The medical device according to claim 10, wherein the distal extension portion includes a distal tube body that is hollow and that includes an inner peripheral surface of the distal extension portion, and a distal covering portion that is tubular, the distal covering portion covering an outer surface of the distal tube body and functioning as the retaining portion.

12. The medical device according to claim 10, wherein at least a part of the linear bodies located in the recess includes an insertion hole, and
the wiring portion penetrates the insertion hole from an inside of the expansion body on a side of the first end with respect to the suspended line portion to expose the first end to the outside of the expansion body.

13. The medical device according to claim 10, wherein the plurality of linear bodies branches or joins at least once or more from the proximal part toward the recess in the expansion body, and
at least a part of the suspended line portion of the wiring portion is located between linear bodies adjacent to each other in the circumferential direction among the plurality of linear bodies.

14. The medical device according to claim 11, wherein the shaft portion includes a tube body including the distal tube body and extending from a distal end to a proximal end of the shaft portion, and a covering portion that is tubular, the covering portion including the distal covering portion and covering the tube body, and
the connection portion of the shaft portion is provided on the covering portion adjacent to a proximal end of the distal covering portion.

15. The medical device according to claim 12, wherein a length of the suspended line portion along a longitudinal direction of the wiring portion is longer than a linear distance between the insertion hole and a section where the suspended line portion is exposed from the retaining portion to the interior of the expansion body.

16. A medical device comprising:
an expansion body including a plurality of linear bodies and expandable and contractible in a radial direction;
a shaft portion that includes a connection portion connected to a proximal part of the expansion body and that is elongated and hollow; and
at least one electrode assembly including an electrode unit and a wiring portion that is elongated and that is connected to the electrode unit, wherein
the expansion body includes a recess recessed radially inward when the expansion body is expanded, and is formed in such a manner that a distance between linear bodies adjacent to each other in a circumferential direction among the plurality of linear bodies constituting the expansion body changes according to expansion and contraction of the expansion body,
the recess includes a bottom located on an innermost side in the radial direction, a proximal-side upright portion extending from a proximal end of the bottom toward a proximal-side top portion on a radially outer side, and a distal-side upright portion extending from a distal end of the bottom toward a distal-side top portion on a radially outer side,
the electrode unit is disposed along the proximal-side upright portion or the distal-side upright portion so as to be exposed to an outside of the expansion body,
the wiring portion of the at least one electrode assembly includes a first end connected to the electrode unit, a second end on an opposite side of the first end, and a suspended line portion disposed between the first end and the second end, and
the suspended line portion of the wiring portion extends from the shaft portion to the proximal-side upright portion or the distal-side upright portion through an interior of the expansion body, and is aerially positioned within the expansion body in an expanded state of the expansion body.

17. The medical device according to claim 16, wherein at least a part of the linear bodies located in the recess includes an insertion hole, and
the wiring portion penetrates the insertion hole from an inside of the expansion body on a side of the first end with respect to the suspended line portion to expose the first end to the outside of the expansion body.

18. The medical device according to claim 17, wherein the insertion hole is provided closer to the proximal-side top portion than the electrode unit disposed at the proximal-side upright portion in the recess.

19. The medical device according to claim 17, wherein the insertion hole is provided closer to the bottom than the electrode unit disposed at the proximal-side upright portion in the recess.

20. The medical device according to claim 17, wherein the insertion hole is provided closer to the bottom than the electrode unit disposed at the distal-side upright portion in the recess.
